(19) United States
(12) Patent Application Publication
(10) Pub. No.: US 2021/0298996 A1
(43) Pub. Date: Sep. 30, 2021

(54) Oral Care Composition and Methods of Use

(71) Applicant: Colgate-Palmolive Company, New York, NY (US)

(72) Inventors: Wei WANG, Plainsboro, NJ (US); Stacey LAVENDER, Chesterfield, NJ (US); Rong DONG, Highland Park, NJ (US)

(21) Appl. No.: 17/214,621

(22) Filed: Mar. 26, 2021

Related U.S. Application Data
(60) Provisional application No. 63/000,742, filed on Mar. 27, 2020.

(51) Int. Cl. A61K 6/20

(52) U.S. Cl. CPC A61K 6/20

(57) ABSTRACT

An oral care composition includes a hydrophobic copolymer, and a polar solvent which can be used to treat and protect dental enamel from damage, for example, cracking or breaking.

### FIELD OF THE INVENTION

The invention generally relates to an oral care composition that includes, e.g., a hydrophobic copolymer, and a polar solvent, at particular amounts and concentrations, which can be used to treat and protect dental enamel from damage, for example, cracking or breaking.

### BACKGROUND

Dental caries and tooth hypersensitivity belong to the most common diseases worldwide. The costs for the public health system caused by them are immense. As result of the loss of enamel or cementum dentinal tubules become exposed to the tooth surface. The consequences are an increased risk of a series of dental diseases/impairments, such as dentin hypersensitivity, caries, and pulp inflammation. Demineralization of enamel and cementum are caused by bacterial bio film formation, especially by the acid-producing Streptococcus mutans.

Teeth contain both enamel and dentin. Dentin is traversed by a network of tubular structures, termed dentinal tubules. These tubules are shielded by the enamel (crown) and the cementum (root), which form a protective layer of the pulp against external physical and chemical influences, like temperature changes and acids, and prevent affection of the nerve protrusions and dentin hypersensitivity. The diameter of the dentinal tubules which protrude into the dentin layer and are open to the dental surface and can vary from 1 and 2.5 μm. Patients suffering from tooth hypersensitivity may have larger number of open dentinal tubules and/or tubules with a larger in diameter than normal.

Unlike other human tissues, tooth enamel is not believed to contain mechanisms for self-protection and rejuvenation. The enamel normally can restore itself by a remineralization process with the necessary minerals and action obtained from saliva. There is a continuous demineralization/remineralization process, which restores the health of the enamel tissue, damaged by the actions described above. There has been the introduction of many new methods for improving the strength of dental enamel and aiding its remineralization. However, as there are no living cells in the mature enamel, the tooth enamel does not contain mechanisms for self-protection and regeneration and, therefore, is essentially a dead tissue.

Accordingly, there is a need for a faster and safer method of hard tissue rejuvenation that provides for accelerated regrowth of an enamel-like layer on the enamel, dentine, cementum or bone without any protection of soft tissues.

### BRIEF SUMMARY

Surprisingly, the present disclosure provides oral care compositions that form a protective layer or coating (e.g., film) for treating and protecting dental enamel from damage, for example, cracking or breaking. The oral care composition (Composition 1.0) which forms a protective layer or coating (e.g., film) over enamel for treating and protecting dental enamel from damage comprises:
a.) from 15.5%-21.0% by wt. of a hydrophobic copolymer comprising 2, 3 or 4 monomers (e.g., 20% 2-propenoic acid, 2-methyl-, 2-methylpropyl ester, polymer with 2-propenoic acid and N-(1,1,3,3-tetramethylbutyl)-2-propenamide); and
b.) greater than 79% by wt of a polar solvent (e.g., about 80% ethanol) (e.g., 80% ethanol).

In another aspect, Composition 1.0 also contemplates the oral care composition of any of the following compositions:
1.1. Composition 1.0, wherein the hydrophobic copolymer is present in an amount from 16.0%-21.0% by wt., relative to the total weight of the composition.
1.2. Composition 1.0 or 1.1, wherein the hydrophobic copolymer is present in an amount from 17.0%-21.0% by wt., relative to the total weight of the composition.
1.3. Composition of any of 1.0-1.2, wherein the hydrophobic copolymer is present in an amount from 18.0%-21.0% by wt., relative to the total weight of the composition.
1.4. Composition of any of 1.0-1.3, wherein the hydrophobic copolymer is selected from the group consisting of: acrylate/octylacrylamide copolymer, VA/Butyl Maleate/Isobornyl Acrylate copolymer, Acrylates/t-Butylacrylamide copolymer, polyvinylpyrrolidone/vinyl acetate copolymer, triacontanyl PVP copolymer, Acrylates/Dimethylaminoethyl Methacrylate copolymer, or mixtures thereof.
1.5. Any of the preceding compositions, wherein the hydrophobic copolymer is 2-propenoic acid, 2-methyl-, 2-methylpropyl ester, polymer with 2-propenoic acid and N-(1,1,3,3-tetramethylbutyl)-2-propenamide (CAS 129702-02-9) (e.g., DERMACRYL® 79 (Nouryon Chemicals Int'l BV)).
1.6. Composition of 1.5, wherein the hydrophobic copolymer is N-(1,1,3,3-tetramethylbutyl)-2-propenamide at about 20% by wt. relative to the total weight of the composition.
1.7. Any of the preceding compositions, wherein the polar solvent is in the amount of 79.5%-99% by wt., relative to total weight of the composition.
1.8. Any of the preceding compositions, wherein the polar solvent is in the amount of 79.5%-89% by wt., relative to total weight of the composition.
1.9. Any of the preceding compositions, wherein the polar solvent is in the amount of 79.5%-85% by wt., relative to total weight of the composition (e.g., 79.5%-80.5% by wt.).
1.10. Any of the preceding compositions, wherein the polar solvent is present in an amount of about 80% by wt. (e.g., 80% by wt.)
1.11. Any of the preceding compositions, wherein the polar solvent is selected from the group consisting of: water, ethanol, acetic acid, and formic acid.
1.12. The composition of 1.11, wherein the polar solvent comprises ethanol (e.g., at 80% by wt.)
1.13. Any of the preceding compositions further comprising a hydrophilic whitening agent.
1.14. The composition of 1.13, wherein the hydrophilic whitening agent comprises a peroxide (e.g., urea peroxide or hydrogen peroxide) or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate), or hydrogen peroxide polymer complexes such as hydrogen peroxide-polyvinyl pyrrolidone polymer complexes).
1.15. Any of the preceding compositions, wherein the composition is an orally acceptable composition.
1.16. Any of the preceding compositions, wherein the composition is a film-forming composition.
1.17. Any of the preceding compositions, wherein the composition is in the form of a liquid (e.g., a flowable composition).
1.18. Any of the preceding compositions, wherein the composition is in the form of a gel (e.g., a viscous liquid dispersion).
1.19. Any of the preceding compositions further comprising a fluoride source selected from: stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof.
1.20. Any of the preceding compositions wherein the pH is between 7.5 and 10.5, e.g., 9.0 to 10.0, e.g., 9.4.
1.21. Any of the preceding compositions further comprising an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophoshpate, monosodium phosphate, pentapotassium triphosphate and mixtures of any of two or more of these, e.g., in an amount of 1-20%, e.g., 2-8%, e.g., ca. 5%>, by weight of the composition.
1.22. Any of the preceding compositions comprising tetrapotassium pyrophosphate, disodium hydrogenorthophoshpate, monosodium phosphate, and pentapotassium triphosphate.
1.23. Any of the preceding compositions further comprising a polyphosphate.
1.24. Any of the preceding compositions further comprising an abrasive or particulate (e.g., silica).
1.25. Any of the preceding compositions comprising silica wherein the silica is used as a thickening agent, e.g., particle silica.
1.26. Any of the preceding compositions further comprising a nonionic surfactant, wherein the nonionic surfactant is in an amount of from 0.5-5%, e.g, 1-2%, selected from poloxamers (e.g., poloxamer 407), polysorbates (e.g., polysorbate 20), polyoxyl hydrogenated castor oil (e.g., polyoxyl 40 hydrogenated castor oil), and mixtures thereof.
1.27. Any of the preceding compositions further comprising one or more antimicrobial agent, wherein antimicrobial agent(s) are selected from the group consisting of: herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, zinc salts (e.g., zinc chloride, zinc lactate, zinc sulfate, zinc citrate and/or zinc oxide), stannous salts (e.g., stannous fluoride, stannous pyrophosphate, and/or stannous chloride), copper salts, iron salts, sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.
1.28. Any of the preceding compositions further comprising a preservative selected from: benzyl alcohol, Methylisothizolinone (“MIT”), Sodium bicarbonate, sodium methyl cocoyl taurate (tauranol), lauryl alcohol, and polyphosphate.
1.29. Any of the preceding compositions comprising polymer films.
1.30. Any of the preceding compositions comprising flavoring, fragrance and/or coloring.
1.31. The composition of 1.30, wherein the flavoring agent is sodium saccharin, sucralose, or a mixture thereof.
1.32. Any of the preceding compositions, wherein the composition comprises a thickening agent selected from the group consisting of carboxyvinyl polymers, carrageenan, hydroxyethyl cellulose and water-soluble salts of cellulose ethers (e.g., sodium carboxymethyl cellulose and sodium carboxymethyl hydroxyethyl cellulose).
1.33. Any of the preceding compositions comprising from 5%-40%, e.g., 10%-35%, e.g., about 15%, 25%, 30%, and 35% water.
1.34. Any of the preceding compositions further comprising an antioxidant, e.g., selected from the group consisting of Co-enzyme Q10, PQQ, Vitamin C, Vitamin E, Vitamin A, BHT, anethole-dithiothione, and mixtures thereof.
1.35. Any of the preceding compositions further comprising an agent that interferes with or prevents bacterial attachment, e.g., ELA or chitosan.
1.36. Any of the preceding compositions further comprising a basic amino acids selected from the group consisting of: arginine, lysine, serine, citrullene, ornithine, creatine, histidine, diaminobutanoic acid, diaminoproprionic acid, salts thereof or combinations thereof.
1.37. Any of the preceding compositions, wherein the oral care composition comprises:
   a.) About 20% 2-propenoic acid, 2-methyl-, 2-methylpropyl ester, polymer with 2-propenoic acid and N-(1,1,3,3-tetramethylbutyl)-2-propenamide) (e.g., DERMACRYL® 79); and
   b.) about 80% ethanol.
1.38. Any of the preceding compositions, wherein the oral care composition consisting (or consisting essentially) of:
   a.) About 20% 2-propenoic acid, 2-methyl-, 2-methylpropyl ester, polymer with 2-propenoic acid and N-(1,1,3,3-tetramethylbutyl)-2-propenamide) (e.g., DERMACRYL® 79); and
   b.) about 80% ethanol.
1.39. Any of the foregoing compositions wherein the oral care composition is a viscous liquid (e.g., gel) (e.g., dental or tooth gel).
1.40. Any of the foregoing compositions wherein the oral care composition is a viscous liquid (e.g., gel) (e.g., dental or tooth gel) that maintains its consistency during storage (e.g., enabling the product to be applied to the tooth surface).
1.41. Any of the foregoing compositions wherein the oral care composition is a gel packaged, and delivered to a patient in need thereof, in a soft applicator dental pen, syringe or brush.
1.42. Any of the foregoing compositions wherein the oral care composition is a gel that is delivered via a syringe and/or dental pen delivery system.
1.43. Any of the foregoing compositions wherein the oral care composition is in the form of a viscoelastic fluid.

Also provided herein is a method of forming any of the disclosed oral care compositions of Compositions 1.0 et seq., comprising adding -propenoic acid, 2-methyl-, 2-methylpropyl ester, polymer with 2-propenoic acid and N-(1,1,3,3-tetramethylbutyl)-2-propenamide and ethanol together.

In yet another aspect, the invention contemplates administering any of Composition 1.0 et seq., in an effective amount to also whiten the surface of a tooth, in addition to treating or preventing damage to the enamel, wherein the contacting of the surface of the tooth with any of the disclosed tooth whitening composition comprises forming a hydrophobic film comprising the hydrophobic polymer and a whitening agent, and wherein the duration of time sufficient to effect whitening of the surface of the tooth comprises diffusing the whitening agent from the hydrophobic film to the tooth surface over a period of time comprising from about 5 minutes to about 24 hours.

In still yet another aspect, the invention contemplates a method (Method 2.0) of treating or preventing enamel damage and/or enamel cracking to a subject in need thereof, wherein the method comprises administering an effective amount of any of the oral care composition of Composition 1.0 et seq, to the enamel of a tooth for duration of time sufficient to treat or protect the enamel from damage or decay.

In another aspect, Method 2.0 contemplates the following methods:
2.1. The Method 2.0, wherein the administration of the oral care composition forms a protective layer or coating on the surface of the tooth enamel, and wherein the protective layer or coating can reduce and/or prevent enamel cracks and/or breaking of the enamel.
2.2. Method 2.0 or 2.1, wherein the protective layer or coating can prevent or reduce enamel crack formation relative to a reference standard.
2.3. Method 2.0 or 2.1, wherein the protective layer or coating can shorten enamel crack duration relative to a reference standard.
2.4. Method 2.0-2.3, wherein the protective layer or coating can reduce, treat, or prevent craze lines, fractured cusp, cracks that extend into the gum line.
2.5. Method of 2.0-2.4, wherein the oral care composition comprises:
   a.) from 15.5%-21.0% by wt. of a hydrophobic copolymer comprising 2, 3 or 4 monomers (e.g., 20% 2-propenoic acid, 2-methyl-, 2-methylpropyl ester, polymer with 2-propenoic acid and N-(1,1,3,3-tetramethylbutyl)-2-propenamide); and
   b.) greater than 79% by wt of a polar solvent (e.g., about 80% ethanol) (e.g., 80% ethanol).
2.6. Any of the preceding methods, wherein the composition comprises a hydrophobic copolymer present in an amount from 16.0%-21.0% by wt., relative to the total weight of the composition.
2.7. Any of the preceding methods, wherein the composition comprises a hydrophobic copolymer present in an amount from 17.0%-21.0% by wt., relative to the total weight of the composition.
2.8. Any of the preceding methods, wherein the composition comprises a hydrophobic copolymer present in an amount from 18.0%-21.0% by wt., relative to the total weight of the composition.
2.9. Any of the preceding methods, wherein the composition comprises a hydrophobic copolymer selected from the group consisting of: acrylate/octylacrylamide copolymer, VA/Butyl Maleate/Isobornyl Acrylate copolymer, Acrylates/t-Butylacrylamide copolymer, polyvinylpyrrolidone/vinyl acetate copolymer, triacontanyl PVP copolymer, Acrylates/Dimethylaminoethyl Methacrylate copolymer, or mixtures thereof.
2.10. Any of the preceding methods, wherein the composition comprises a hydrophobic copolymer, wherein the hydrophobic copolymer is 2-propenoic acid, 2-methyl-, 2-methylpropyl ester, polymer with 2-propenoic acid and N-(1,1,3,3-tetramethylbutyl)-2-propenamide (CAS 129702-02-9) (e.g., DERMACRYL® 79 (Nouryon Chemicals Int'l BV)).
2.11. The Method of 2.09, wherein the hydrophobic copolymer is N-(1,1,3,3-tetramethylbutyl)-2-propenamide at about 20% by wt. relative to the total weight of the composition.
2.12. Any of the preceding methods, wherein the oral care composition comprises a polar solvent in the amount of 79.5%-99% by wt., relative to total weight of the composition.
2.13. Any of the preceding methods, wherein the oral care composition comprises a polar solvent in the amount of 79.5%-89% by wt., relative to total weight of the composition.
2.14. Any of the preceding methods, wherein the oral care composition comprises a polar solvent in the amount of 79.5%-85% by wt., relative to total weight of the composition (e.g., 79.5%-80.5% by wt.).
2.15. Any of the preceding methods, wherein the oral care composition comprises a polar solvent in the amount of about 80% by wt. (e.g., 80% by wt.).
2.16. Any of the preceding methods, wherein the composition is in the form of a liquid (e.g., a flowable composition).
2.17. Any of the preceding methods, wherein the composition is in the form of a gel (e.g., a viscous liquid dispersion).
2.18. Any of the preceding methods, wherein the subject is at elevated or increased risk of developing enamel cracks (e.g., based upon prior damage, and wherein the composition prevents further damage).
2.19. Any of the preceding methods, wherein the subject has weakened and/or structurally compromised enamel (e.g., there is a pre-existing crack).
2.20. Any of the preceding methods, wherein the oral care composition comprises:
   a.) About 20% 2-propenoic acid, 2-methyl-, 2-methylpropyl ester, polymer with 2-propenoic acid and N-(1,1,3,3-tetramethylbutyl)-2-propenamide) (e.g., DERMACRYL® 79); and
b.) about 80% ethanol.

In yet a further aspect, the invention contemplates a Delivery System (Delivery System 1) for administration of any of Composition 1, et seq to a patient in need thereof. In one aspect the delivery system comprises any of Composition 1, et seq, wherein the composition is a gel. In another aspect, the delivery system comprises a syringe for administration of the composition of any of Composition 1, et seq (e.g., wherein the syringe is used by a professional). In another aspect the delivery system comprises both a syringe and a dental pen for administration of any of Composition 1, et seq.

Further areas of applicability of the present invention will become apparent from the detailed description provided hereinafter. It should be understood that the detailed description and specific examples, while indicating some preferred aspects of the invention, are intended for purposes of illustration only and are not intended to limit the scope of the invention.

### DETAILED DESCRIPTION

The following description of various preferred aspect(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The term “hydrophobic” as applied to polymers and copolymers and employed herein refers to an organic polymer or copolymer which is substantially non-aqueous and having a water solubility of less than 2% g/g water at a pH of 8 and below.

The term “hydrophilic” is used herein consistent with its standard meaning of having affinity for water.

As used herein, “film-forming composition” refers to a material or combination of materials that may precipitate out of solution, for example, as the composition dries upon application to a surface (e.g., as solvent evaporates away), thereby leaving behind a film of the precipitated material or combination of materials.

As used herein, “orally acceptable” refers to a material or combination of materials that are safe for use in the compositions of the present disclosure, commensurate with a reasonable benefit/risk ratio, with which the whitening agent, and other desired active ingredients may be associated while retaining significant efficacy.

As used herein, “enamel cracks” or “cracked enamel” can be used interchangeably. “Enamel cracks” can include, but are not limited to: craze lines, fractured cusp, cracks that extend into the gum line, and vertical root fractures.

As used herein, “craze lines” are intended to mean small cracks in the enamel. “Fractured cusp” is intended to mean a crack that generally occurs around a dental filling but does not affect the pulp of the tooth. “Cracks that extend into the gum line” is intended to mean a vertical crack that extends into the gum line. A “split tooth” is intended to mean a crack that travels from its surface to below the gum line. A “vertical root fracture” is intended to mean the type of crack that begins below the gum line and travels upward.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, “orally acceptable” refers to a material that is safe and palatable at the relevant concentrations for use in an oral care formulation, such as a mouthwash or dentifrice.

As used herein, “orally acceptable carrier” refers to any vehicle useful in formulating the oral care compositions disclosed herein. The orally acceptable carrier is not harmful to a mammal in amounts disclosed herein when retained in the mouth, without swallowing, for a period sufficient to permit effective contact with a dental surface as required herein. In general, the orally acceptable carrier is not harmful even if unintentionally swallowed. Suitable orally acceptable carriers include, for example, one or more of the following: water, a thickener, a buffer, a humectant, a surfactant, an abrasive, a sweetener, a flavorant, a pigment, a dye, an anti-caries agent, an anti-bacterial, a whitening agent, a desensitizing agent, a vitamin, a preservative, an enzyme, and mixtures thereof.

As used herein, “viscoelastic fluid” refers to a complex fluid that exhibits mechanical properties that are both elastic (solid-like, e.g., rubber) and viscous (liquid-like or flowable, e.g., water). A viscoelastic fluid composition may deform and flow under the influence of an applied shear stress (e.g., shaking or swishing in the mouth), but when the stress is removed, the composition will recover the deformation.

### Hydrophobic Copolymer

In various aspects, the oral care compositions disclosed herein comprise a hydrophobic copolymer. The hydrophobic copolymer may be selected from a carboxylated acrylic copolymer such as a copolymer of octylacrylamide and one or more monomers comprising acrylic acid, methacrylic acid, or one or more simple esters thereof. In one aspect the hydrophobic copolymer of the tooth whitening compositions may be an acrylate/octylacrylamide copolymer. In yet another aspect, the hydrophobic copolymer preferably comprises a copolymer (e.g., 2, 3 or 4 monomers), for example, 2-Propenoic acid, 2-methyl-, 2-methylpropyl ester; polymer with 2-propenoic acid; and N-(1,1,3,3-tetramethylbutyl)-2-propenamide (CAS 129702-02-9) which is available as DERMACRYL® 79 (Nouryon Chemicals Int'l BV).

In at least one further aspect, Composition 1.0 et seq, or Method 2.0 et seq., include a hydrophobic polymer that may be a copolymer of octylacrylamide and one or more monomers, where the one or more monomers may include one or more of acrylic acid, methacrylic acid, and any one or more simple esters thereof. In yet another aspect, the hydrophobic polymer of Composition 1.0 et seq, or Method 2.0 et seq. may be a polymer formed from octylacrylamide, t-butylaminoethyl methacrylate, and one or more monomers of acrylic acid, methacrylic acid, or any one or more simple esters thereof. Illustrative carboxylated acrylic copolymers may be or include, but are not limited to, those sold under the trade names DERMACRYL®, AMPHOMER®, BALANCE®, and VERSACRYL®, which are commercially available from Nouryon Chemicals Int'l BV (e.g., 2-Propenoic acid, 2-methyl-, 2-[(1,1-dimethylethyl)amino]ethyl ester, polymer with methyl 2-methyl-2-propenoate, 1,2-propanediol mono(2-methyl-2-propenoate), 2-propenoic acid and N-(1,1,3,3-tetramethylbutyl)-2-propenamide (CAS No.: (70801-07-9); e.g., 2-propenoic acid, 2-methyl-, 2-methylpropyl ester, polymer with 2-propenoic acid and N-(1,1,3,3-tetramethylbutyl)-2-propenamide (CAS 129702-02-9)).

For example, the carboxylated acrylic copolymers may be or include, but are not limited to, AMPHOMER® 4961, AMPHOMER® HC, DERMACRYL® 2.0, RESYN™ XP, a hydrophobic copolymer selected from octylacrylamide/acrylates/butylaminoethyl methacrylate copolymer, such as AMPHOMER® LV-71, AMPHOMER®, AMPHOMER® EDGE™, BALANCE® 47, and combinations thereof, all of which are commercially available from Nouryon Chemicals Int'l BV. The hydrophobic copolymer may be selected from VA/butyl maleate/isobornyl acrylate copolymer, such as ADVANTAGE™ PLUS from Ashland Global Specialty Chemicals Inc. of Covington, Ky. The hydrophobic copolymer may be selected from acrylates/t-butylacrylamide copolymer, such as ULTRAHOLD® STRONG and ULTRAHOLD®8 from BASF SE of Ludwigshafen, Germany. The hydrophobic copolymer may be selected from acrylates/dimethylaminoethyl methacrylate copolymer, such as the EUDRAGIT® range of polymers from Evonik Industries of Essen, Germany, such as EUDRAGIT®E100, EUDRAGIT® E PO, EUDRAGIT® RS 100, EUDRAGIT® RS PO, EUDRAGIT® RL PO, EUDRAGIT® RL 100, or the like, and combinations thereof. The hydrophobic copolymer may be selected from polyvinylpyrrolidone/vinyl acetate, such as the PVP/VA series of polymers from Ashland Global Specialty Chemicals Inc. of Covington, Ky. The hydrophobic copolymer may be selected from triacontanyl PVP, such as GANEX™ WP-660 from Ashland Global Specialty Chemicals Inc. of Covington, Ky. The hydrophobic copolymer may be selected from at least one of octylacrylamide/acrylates/butylaminoethyl methacrylate copolymer, VA/butyl maleate/isobornyl acrylate copolymer, acrylates/t-butylacrylamide copolymer, polyvinylpyrrolidone/vinyl acetate copolymer, triacontanyl PVP copolymer, acrylates/dimethylaminoethyl methacrylate copolymer, or mixtures thereof. In an preferred implementation, the hydrophobic polymer may be a copolymer of 2-Propenoic acid, 2-methyl-, 2-methylpropyl ester, polymer with 2-propenoic acid and N-(1,1,3,3-tetramethylbutyl)-2-propenamide or 2-propenoic acid, 2-methyl-, 2-methylpropyl ester, 2-propenoic acid, N-(1,1,3,3-tetramethylbutyl)-2-propenamide copolymer (CAS 129702-02-9). For example, the hydrophobic polymer may be or include, but is not limited to, DERMACRYL® 79, which is commercially available from AkzoNobel Company, Surface Chemistry of Amsterdam, Netherlands.

### Polar Solvent

In various implementations, the tooth cleaning composition disclosed herein includes a polar solvent. The polar solvent is selected such that it is capable of at least partially dissolving the hydrophobic copolymer. The polar solvent may comprise glycerin, propylene glycol, alcohol, ethyl acetate, other esters and aldehydes or water.

### Amino Acid

In some embodiments, any of Compositions 1.0 et seq can include a basic or neutral amino acid. The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

For example, basic amino acids include, but are not limited to, arginine, lysine, serine, citrullene, ornithine, creatine, histidine, diaminobutanoic acid, diaminoproprionic acid, salts thereof or combinations thereof. In a particular embodiment, the basic amino acids are selected from arginine, citrullene, and ornithine.

In certain embodiments, the basic amino acid is arginine, for example, L-arginine, or a salt thereof.

In another aspect, the compositions of the invention (e.g., any of Compositions 1.0 et seq) can include a neutral amino acid, which can include, but are not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

The compositions of the invention are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

### Fluoride Ion Source

The oral care composition and methods described herein may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., each of which are incorporated herein by reference. Representative fluoride ion sources used with the present invention (e.g., Composition 1.0 et seq.) include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. Where the formulation comprises calcium salts, the fluoride salts are preferably salts wherein the fluoride is covalently bound to another atom, e.g., as in sodium monofluorophosphate, rather than merely ionically bound, e.g., as in sodium fluoride.

### Surfactants

The oral care composition and methods described herein may in some embodiments contain anionic surfactants, e.g., the Compositions of Composition 1.0, et seq and Method 2.0 et seq., for example, water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOS0₃X, wherein m is 6-16, e.g., 10, n is 1-6, e.g., 2, 3 or 4, and X is Na or, for example sodium laureth-2 sulfate (CH₃(CH₂)₁₀CH₂(OCH₂CH₂)₂OS0₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C₆₋₃₀ alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., 1.5%.

In another embodiment, cationic surfactants useful in the present invention can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants of Composition 1.0, et seq. and/or Method 2.0 et seq, that can be used in the compositions of the invention can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the invention comprises a nonionic surfactant selected from polaxamers (e.g., polaxamer 407), polysorbates (e.g., polysorbate 20), polyoxyl hydrogenated castor oils (e.g., polyoxyl 40 hydrogenated castor oil), and mixtures thereof.

In still another embodiment amphoteric surfactants can be used. Suitable amphoteric surfactants, without limitation, are derivatives of C₈₋₂₀ aliphatic secondary and tertiary amines having an anionic group such as carboxylate, sulfate, sulfonate, phosphate or phosphonate. A suitable example is cocoamidopropyl betaine. One or more surfactants are optionally present in a total amount of 0.01 weight % to 10 weight %, for example, from 0.05 weight % to 5 weight % or from 0.1 weight % to 2 weight % by total weight of the composition.

The surfactant or mixtures of compatible surfactants can be present in the compositions of the present invention in 0.1% to 5%, in another embodiment 0.3% to 3% and in another embodiment 0.5% to 2% by weight of the total composition.

### Flavoring Agents

The oral care composition and methods of the invention may also include a flavoring agent. Flavoring agents which are used in the practice of the present invention include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of 0.01 to 1% by weight.

### Chelating and Anti-Calculus Agents

The oral care composition and methods of the invention also may include one or more chelating agents able to complex calcium found in the cell walls of the bacteria. Binding of this calcium weakens the bacterial cell wall and augments bacterial lysis.

Another group of agents suitable for use as chelating or anti-calculus agents in the present invention are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide at least 0.5 wt. % pyrophosphate ions, 0.9-3 wt. %. The pyrophosphates also contribute to preservation of the compositions by lowering water activity.

### Polymers

The oral care composition and methods of the invention also optionally include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139 (M.W. 500,000), AN 1 19 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

Another useful class of polymeric agents includes polyamino acids, particularly those containing proportions of anionic surface-active amino acids such as aspartic acid, glutamic acid and phosphoserine, as disclosed in U.S. Pat. No. 4,866,161 Sikes et al., incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, carrageenan, hydroxyethyl cellulose and water-soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

### Abrasives

The oral care composition and methods of the invention can further include one or more abrasives. Natural calcium carbonate is found in rocks such as chalk, limestone, marble and travertine. It is also the principle component of egg shells and the shells of mollusks. The natural calcium carbonate abrasive of the invention is typically a finely ground limestone which may optionally be refined or partially refined to remove impurities. For use in the present invention, the material has an average particle size of less than 10 microns, e.g., 3-7 microns, e.g. about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004% by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8=4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high water absorption, e.g., at least 25 g/100 g, e.g. 30-70 g/100 g. Examples of commercially available products suitable for use in the present invention include, for example, Carbolag® 15 Plus from Lagos Industria Quimica.

In certain embodiments the invention may comprise additional calcium-containing abrasives, for example calcium phosphate abrasive, e.g., tricalcium phosphate (Ca₃(P0₄)₂), hydroxyapatite (Ca₁₀(P0₄)₆(OH)₂), or dicalcium phosphate dihydrate (CaHPO₄. 2H₂0, also sometimes referred to herein as DiCal) or calcium pyrophosphate, and/or silica abrasives, sodium metaphosphate, potassium metaphosphate, aluminum silicate, calcined alumina, bentonite or other siliceous materials, or combinations thereof. Any silica suitable for oral care compositions may be used, such as precipitated silicas or silica gels. For example synthetic amorphous silica. Silica may also be available as a thickening agent, e.g., particle silica. For example, the silica can also be small particle silica (e.g., Sorbosil AC43 from Ineos Silicas, Warrington, United Kingdom). However the additional abrasives are preferably not present in a type or amount so as to increase the RDA of the dentifrice to levels which could damage sensitive teeth, e.g., greater than 130.

### Enzymes

The oral care composition and methods of the invention may also optionally include one or more enzymes. Useful enzymes include any of the available proteases, glucanohydrolases, endoglycosidases, amylases, mutanases, lipases and mucinases or compatible mixtures thereof In certain embodiments, the enzyme is a protease, dextranase, endoglycosidase and mutanase. In another embodiment, the enzyme is papain, endoglycosidase or a mixture of dextranase and mutanase. Additional enzymes suitable for use in the present invention are disclosed in U.S. Pat. No. 5,000,939 to Dring et al., U.S. Pat. Nos. 4,992,420; 4,355,022; 4,154,815; 4,058,595; 3,991,177; and 3,696,191 all incorporated herein by reference. An enzyme of a mixture of several compatible enzymes in the current invention constitutes 0.002% to 2.0% in one embodiment or 0.05% to 1.5% in another embodiment or in yet another embodiment 0.1% to 0.5%.

### Water

Water is present in the oral care composition and methods of the invention. Water, employed in the preparation of commercial oral compositions should be deionized and free of organic impurities. Water commonly makes up the balance of the compositions and includes 5% to 45%, e.g., 10% to 20%, e.g., 25-35%, by weight of the oral compositions. This amount of water includes the free water which is added plus that amount which is introduced with other materials such as with sorbitol or silica or any components of the invention. The Karl Fischer method is a one measure of calculating free water.

### Humectants

Within certain embodiments of the oral compositions and methods of the invention, it is also desirable to incorporate a humectant to reduce evaporation and also contribute towards preservation by lowering water activity. Certain humectants can also impart desirable sweetness or flavor to the compositions. The humectant, on a pure humectant basis, generally includes 15% to 70% in one embodiment or 30% to 65% in another embodiment by weight of the composition.

Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the compositions herein.

The present invention in its method aspect involves applying to the oral cavity a safe and effective amount of the compositions described herein.

The compositions and methods according to the invention are useful to a method to protect the teeth by facilitating repair and remineralization, in particular to reduce or inhibit formation of dental caries, reduce or inhibit demineralization and promote remineralization of the teeth, reduce hypersensitivity of the teeth, and reduce, repair or inhibit early enamel lesions, e.g., as detected by quantitative light-induced fluorescence (QLF) or electronic caries monitor (ECM).

Quantitative Light-induced Fluorescence is a visible light fluorescence that can detect early lesions and longitudinally monitor the progression or regression. Normal teeth fluoresce in visible light; demineralized teeth do not or do so only to a lesser degree. The area of demineralization can be quantified and its progress monitored. Blue laser light is used to make the teeth auto fluoresce. Areas that have lost mineral have lower fluorescence and appear darker in comparison to a sound tooth surface. Software is used to quantify the fluorescence from a white spot or the area/volume associated with the lesion. Generally, subjects with existing white spot lesions are recruited as panelists. The measurements are performed in vivo with real teeth. The lesion area/volume is measured at the beginning of the clinical. The reduction (improvement) in lesion area/volume is measured at the end of 6 months of product use. The data is often reported as a percent improvement versus baseline.

Electrical Caries Monitoring is a technique used to measure mineral content of the tooth based on electrical resistance. Electrical conductance measurement exploits the fact that the fluid-filled tubules exposed upon demineralization and erosion of the enamel conduct electricity. As a tooth loses mineral, it becomes less resistive to electrical current due to increased porosity. An increase in the conductance of the patient's teeth therefore may indicate demineralization. Generally, studies are conducted of root surfaces with an existing lesion. The measurements are performed in vivo with real teeth. Changes in electrical resistance before and after 6-month treatments are made. In addition, a classical caries score for root surfaces is made using a tactile probe. The hardness is classified on a three-point scale: hard, leathery, or soft. In this type of study, typically the results are reported as electrical resistance (higher number is better) for the ECM measurements and an improvement in hardness of the lesion based on the tactile probe score.

Test methods for the desensitizing properties of the compositions described herein, uses the method described in U.S. Pat. No. 5,589,159, the disclosure of which is incorporated by reference herein in its entirety. This method measures the hydraulic conductance of materials, providing an objective reduction in fluid flow that correlates with reduction in fluid flow in dentinal tubules. In this method, intact human molars free from caries and restorations are sectioned perpendicularly to the long axis of the tooth with a metallurgical saw to form thin sections, or discs, from about 0.4 to about 0.8 mm thick. Sections containing dentin and free of enamel were selected for testing and then etched with citric acid solution to remove the smear layer. Each disc was mounted into a split chambered device described in J. Dent. Research, 57: 187 (1978) which is a special leak-proof chamber connected to a pressurized fluid reservoir containing a tissue culture fluid. By using a mixture of pressurized nitrogen and carbon dioxide gas, the fluid can be made at physiological pH. To further ensure accuracy, the discs were wetted with artificial saliva (phosphate buffer saline, PBS) to approximate intra-oral conditions. The apparatus includes a glass capillary tube attached to a flow sensor (FLODEC, DeMarco Engineering SA, Geneva). An air bubble is injected into the glass capillary tube. By measuring the displacement of the bubble as a function of time, fluid flow through the dentin disc can be measured. Fluid flow is equivalent to the dentin permeability.

The Compositions of the Invention are thus useful in a method to reduce early lesions of the enamel (as measured by QLF or ECM) relative to a composition lacking effective amounts of fluorine and/or arginine.

The compositions and methods according to the invention (e.g., Composition 1.0 et seq or Method 2.0 et seq.) can be incorporated into oral compositions for the care of the mouth and teeth such as toothpastes, transparent pastes, gels, mouth rinses, sprays and chewing gum.

In yet another aspect, the oral care compositions disclosed herein may be used in the manufacture of an oral care product may have the additional benefit of protecting the teeth from staining, bacteria, or for whitening teeth.

In still another aspect, the Compositions of Composition 1.0 et seq and Method 2.0 et seq may be prepared in the form of a flowable composition, such as a liquid, or as a viscous liquid dispersion, such as a gel. In an implementation, the tooth whitening compositions disclosed herein may be used in a method of protecting the teeth from staining or bacteria, for example, by applying any of the foregoing compositions to the teeth.

The oral care composition of Composition 1.0 et seq or Method 2.0 et seq., may be introduced to the mouth directly, such as by rinsing (e.g., the user swirls the composition in the mouth like mouthwash). That is, preferably, application of any of the tooth whitening compositions leaves behind a film comprising the hydrophobic copolymer on the teeth.

Alternatively, the oral care compositions of Composition 1.0 et seq or Method 2.0 et seq may be applied directly onto the user's teeth with an applicator, such as by painting the teeth with an applicator brush or an applicator pen. Accordingly, in at least one implementation, a package comprises any of the foregoing tooth whitening compositions together with an applicator for applying the composition to the teeth.

Alternatively, the oral care compositions of Composition 1.0 et seq or Method 2.0 et seq may be applied directly onto the user's teeth via placement of the composition on a substrate, such as a strip or mouth-tray. For example, any one of the tooth whitening compositions described above can be poured or spread onto a surface of the substrate and then the substrate may be placed in the user's mouth to place the composition in contact with the tooth surface. The substrate may remain in place or may be removed, leaving at least some of the composition on the surface of at least one tooth in the user's mouth.

### EXAMPLES

### Example 1

An exemplary oral care composition according to the formulations described herein is prepared. The oral care composition used in the Examples described herein is as follows:

TABLE 1

| Formula 1 | % |
|---|---|
| DERMACRYL ® 79 | 20.0% |
| EtOH | 80.0% |
| Total | 100.00% |

The exemplary oral care composition is prepared by combining the ingredients/components according to Table 1. Specifically, the ingredients/components were combined or otherwise contacted with one another in a spin mix jar and mixed at about 3540 rpms for about 5 minutes until a homogenous suspension was obtained.

Prevention and/or Treatment of Enamel Cracking

Enamel blocks are obtained from sound bovine incisors without defects. The labial surface of bovine teeth are cut to obtain an enamel specimen (˜3×3×2 mm). The enamel layer is ˜1 mm thick and the dentin left in the specimen is ˜1 mm thick. The enamel blocks are then grinded and polished.

Bovine enamel blocks are kept in 4 ml artificial saliva (AS). When the cracks are ready to be generated, an enamel block is taken out of AS, and kimwipes are used to gently dry the surface. Subsequently, an indentation is made on the left half of the enamel surface in order to generate one or more crack(s). The cracks can be produced at 1000-, 500- or 300-gram force.

After each indention, the enamel block is observed to see whether there are one or more crack(s) next to the indentation. Cracks can also be measured by length.

The enamel block is placed back into AS, and incubated for a relatively short period of time, and then taken out again. The block is gently wiped to dry the surface, and then a further indentation is made. Care is taken so that the enamel block is not dehydrated, possibly subjecting it to additional cracking.

Where the enamel block generates cracks on the left side on enamel surface, Formula 1 (20% Dermacryl 79, 80% EtOH) is applied on the right half of the enamel surface. Formula 1 is allowed to dry for several minutes. Once dry, an indentation is made on the right (coated) side of enamel. The coating is removed using ethanol. Once more, the block is observed to determine if cracks form on the right side of the enamel surface.

Note, force is applied on enamel surface using a Buehler Microhardness Tester (Knoop hardness, 500 grams load or higher). As shown in Table 2 below, the enamel that is treated with Formula 1 demonstrates enamel crack prevention benefits.

TABLE 2

| | Untreated Portion of Enamel | Treated Portion of Enamel |
|---|---|---|
| | Percent of incidents that | Percent of incidents that |
| | Cracks were generated on | Cracks were generated on |
| Block | un-coated enamel out of | coated enamel out of |
| number | total indentation times | total indentation times |
| #3 | 100% | 60% |
| #4 | 100% | 0% |
| #8 | 100% | 60% (40% are tiny cracks) |
| #9 | 100% | 20% |

1. An oral care composition that forms a protective layer or coating over enamel for treating and protecting dental enamel from damage and/or cracking comprising:
a.) from 15.5%-21.0% by wt. of a hydrophobic copolymer comprising 2, 3 or 4 monomers; and
b.) greater than 79% by wt of a polar solvent.

2. The oral care composition of claim 1, wherein the hydrophobic copolymer is present in an amount from 16.0%-21.0% by wt., relative to the total weight of the composition.

3. The oral care composition of claim 1, wherein the hydrophobic copolymer is present in an amount from 17.0%-21.0% by wt., relative to the total weight of the composition.

4. The oral care composition of claim 1, wherein the hydrophobic copolymer is present in an amount from 18.0%-21.0% by wt., relative to the total weight of the composition.

5. The oral care composition of claim 1, wherein the hydrophobic copolymer is selected from the group consisting of: acrylate/octylacrylamide copolymer, VA/Butyl Maleate/Isobornyl Acrylate copolymer, Acrylates/t-Butylacrylamide copolymer, polyvinylpyrrolidone/vinyl acetate copolymer, triacontanyl PVP copolymer, Acrylates/Dimethylaminoethyl Methacrylate copolymer, or mixtures thereof.

6. The oral care composition of claim 1, wherein the hydrophobic copolymer is 2-propenoic acid, 2-methyl-, 2-methylpropyl ester, polymer with 2-propenoic acid and N-(1,1,3,3-tetramethylbutyl)-2-propenamide.

7. The oral care composition of claim 1, wherein the hydrophobic copolymer is N-(1,1,3,3-tetramethylbutyl)-2-propenamide at about 20% by wt. relative to the total weight of the composition.

8. The oral care composition of claim 1, wherein the polar solvent is in the amount of 79.5%-99% by wt., relative to total weight of the composition.

9. The oral care composition of claim 1, wherein the polar solvent is in the amount of 79.5%-89% by wt., relative to total weight of the composition.

10. The oral care composition of claim 1, wherein the polar solvent is in the amount of 79.5%-85% by wt., relative to total weight of the composition.

11. The oral care composition of claim 1, wherein the polar solvent is ethanol, wherein the ethanol is present in an amount of about 80% by wt., relative to the total weight of the composition.

12. A method of treating or preventing enamel damage and/or enamel cracking to a subject in need thereof, wherein the method comprises administering an effective amount of the oral care composition of claim 1, wherein the administration of the oral care composition forms a protective layer on the surface of the tooth enamel, and wherein the protective layer can reduce and/or prevent enamel cracks and/or breaking of the enamel.

13. The method of claim 12, wherein the protective layer can prevent or reduce enamel crack formation relative to a reference standard.

14. The method of claim 13, wherein the protective layer can shorten enamel crack duration relative to a reference standard.

15. The method of claim 12, wherein the protective layer can reduce, treat, or prevent craze lines, fractured cusp, cracks that extend into the gum line.

16. (canceled)

17. The method of claim 12, wherein the oral care composition comprises a polar solvent in the amount of about 80% by wt.

18. The method of claim 12, wherein the composition is in the form of a liquid.

19. The method of claim 12, wherein the composition is in the form of a gel (e.g., a viscous liquid dispersion).

20. The method of claim 12, wherein the subject is at elevated or increased risk of developing enamel cracks.

21. The method of claim 12, wherein the oral care composition comprises:
a. About 20% 2-propenoic acid, 2-methyl-, 2-methylpropyl ester, polymer with 2-propenoic acid and N-(1,1,3,3-tetramethylbutyl)-2-propenamide); and
b. about 80% ethanol.
